(19) United States
(12) Patent Application Publication
(10) Pub. No.: US 2004/0158122 A1
(43) Pub. Date: Aug. 12, 2004

(54) Intra-vaginal device for preventing female stress incontinence

(76) Inventor: Bernard Guerquin, Saint Avold (FR)

Correspondence Address: YOUNG & THOMPSON, 745 SOUTH 23RD STREET 2ND FLOOR, ARLINGTON, VA, 22202

(21) Appl. No.: 10/476,669

(22) PCT Filed: May 7, 2002
(86) PCT No.: PCT/FR02/01558

(30) Foreign Application Priority Data
May 7, 2001 (FR) 01/06063

(51) Int. Cl. A61F 2/00

(52) U.S. Cl. 600/29

(57) ABSTRACT

The invention concerns a device (10) for preventing female stress incontinence designed to be arranged longitudinally inside the vagina. The device comprises a proximal part (22) extending between a first free end (22a) and a second end (22b), designed so as to be substantially undeformable under pressure, and adapted to be placed in the sub-urethral zone of the vagina, and a distal part (24) extending the second end (22b) of the proximal part (22), made of a material reversibly deformable under pressure, designed to be placed in a sub-bladder zone of the vagina and having a height not more than 80% of the height of said proximal part (22).

The invention relates to a device for preventing female urinary stress incontinence to be arranged in a longitudinal manner in the vagina.

Provision is made in currently available devices for the urethral/vesical junction to be relieved by a prosthesis which is arranged in the vagina facing this junction.

These prostheses are not always very effective or, in some cases, are completely ineffective. Sometimes the prostheses of the prior art also lead to difficulties in terms of positioning by the user herself and can require a medical follow-up, at least during the period of adaptation to a prosthesis which meets the expectations of the user in terms of comfort and results.

The problem addressed by the present invention is to overcome these disadvantages by providing a simple prosthetic device which can be produced according to a method, which is easy to carry out, and which the user will be able to position alone in the correct manner and be able to use in particular over short periods, for example, during sporting activities.

This problem is solved according to the present invention by a device which comprises a proximal portion which extends between a first free end and a second end, which is produced so as to be substantially non-deformable under pressure and which is intended to be positioned in the sub-urethral region of the vagina, and a distal portion which extends the second end of the proximal portion, which is produced from a material which is deformable under pressure in a reversible manner, which is intended to be positioned in the sub-vesical region of the vagina and which has a height which is at most 80% of the height of said proximal portion.

In a preferred manner, said distal portion has a height of between 40 and 60% of the height of said proximal portion, and advantageously in the order of 50% of the height of said proximal portion.

Owing to this specific configuration in two portions, the morphological and functional differences of the front sub-urethral region and the rear sub-vesical region of the anterior vaginal wall are taken into consideration, as will be explained in greater detail below.

The major role of the weakening of the strength of the anterior wall of the vagina in the region located below the urethra has been noted in female urinary stress incontinence, the collapse of the anterior wall of the vagina in the region located under the bladder itself having an effect which is instead protective and which must be respected.

The smaller height of the distal portion is significant because it allows the deformation of the vaginal wall in the sub-vesical portion thereof to be respected, whereas the greater height of the proximal portion improves the mechanical strength of the anterior wall of the vagina in the sub-urethral portion thereof.

Said distal portion preferably has a width which is at least 1.5 times the width of said proximal portion.

Since this configuration with the distal portion being widened takes into consideration the greater width of the vagina in the depth thereof, it ensures the correct positioning of the device in the vagina whatever the initial orientation of the device when it is introduced.

The distal portion preferably has a width which is greater than the height thereof. This allows the larger diameter of the vagina in the sub-vesical (rear) region thereof relative to the sub-urethral (front) region thereof to be taken into consideration without having to use a distal portion which is excessively large and therefore difficult to introduce into the front region of the vagina.

Depending on the material(s) used in the production of this device, which will be biocompatible at least in the external portion thereof, the device can also fulfil the conventional function of a periodic or sanitary intra-vaginal tampon which is introduced into the vagina during menstruation.

In order to reconcile the functional necessities with the female anatomical dimensions, the proximal portion preferably has a length of between 1.5 and 2.5 cm, advantageously in the order of 2 cm, and the distal portion preferably has a length of between 2 and 5 cm, advantageously in the order of 3 cm.

In a preferable but non-limiting manner, the proximal portion has a cylindrical shape of circular cross-section or a frustoconical shape which widens from the first end towards the second end.

According to one possible variant, the proximal portion has a core which is produced from a hard material which is surrounded by a flexible casing which is produced from a biocompatible material. In this case, said core preferably has, in the longitudinal direction, an elongate shape, either cylindrical with a longitudinal axis and circular cross-section or oblong.

According to another preferred variant, the distal portion is formed by two similar tubular members which are arranged side by side over the length thereof in a connected or separate manner.

In this manner, a distal portion is produced which is wider than it is high and which is adapted to the shape of the sub-vesical region of the vagina and which also prevents the spontaneous expulsion of the treatment element out of the vagina because the distal portion is wider than the width at rest of the sub-urethral region of the vagina.

According to one embodiment, the distal portion is produced from foam, such as a polyurethane foam, or from any other material having similar features in terms of flexibility, resilience and biological compatibility.

According to a preferred embodiment, the device further comprises removal means for the device which are mounted on the first end of the proximal portion, for example, a string, which it is simply necessary to pull in order to draw the device out of the vagina.

According to another preferred embodiment, the device further comprises insertion means which facilitate the positioning of the device in the vagina. Said insertion means are advantageously in the form of an applicator which comprises a hollow tube which is able to contain the proximal and distal portions and a pusher which can slide in a telescopic manner in said tube in order to carry the proximal and distal portions into the vagina, facing the sub-urethral and sub-vesical regions of the anterior wall of the vagina, respectively.

Several embodiments will now be described and illustrated below. It should be noted that the description and the drawings are given purely by way of non-limiting example.

Reference will be made to the appended drawings, in which:

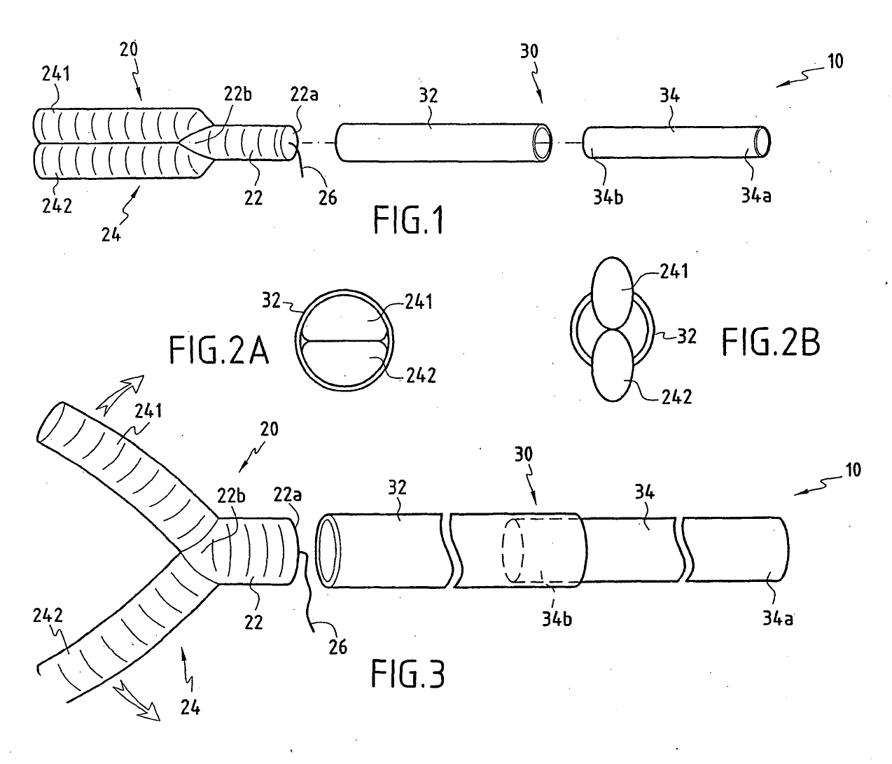

FIG. 1 is an exploded longitudinal view of the various elements which form a device for preventing female urinary stress incontinence which is the subject-matter of the present invention;

FIG. 2A is an illustration in the transverse direction according to direction 1 of FIG. 1 when the distal portion is inserted into the hollow tube of the insertion means;

FIG. 2B is an illustration in the transverse direction according to direction 1 of FIG. 1 (the distal portion has left the hollow tube of the insertion means);

FIG. 3 is a view similar to that of FIG. 1 for a first variant of the device according to the invention;

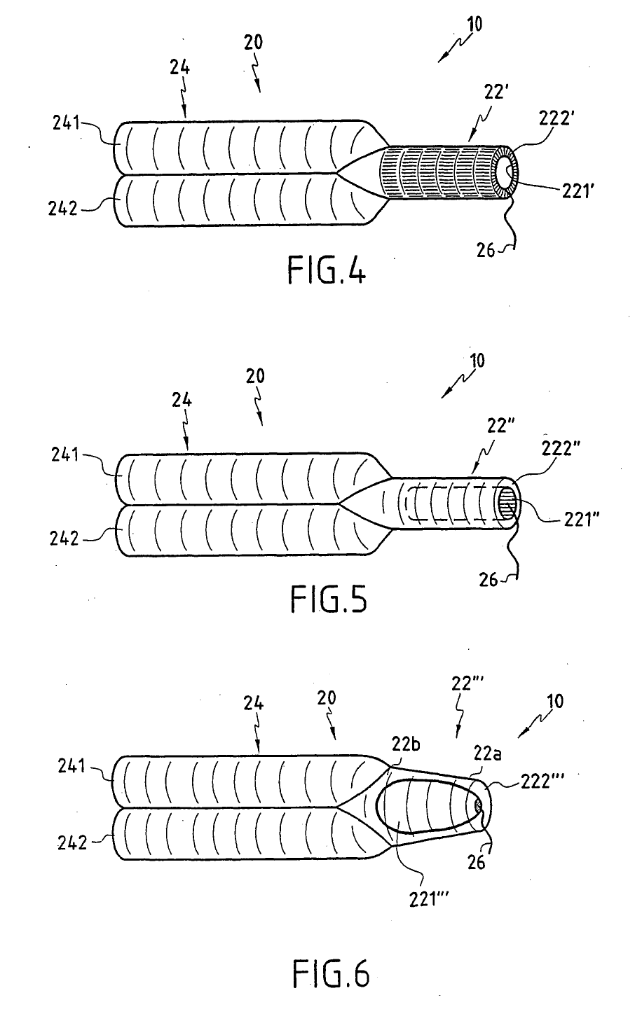

FIG. 4 is a longitudinal projection of a second variant of the device which is the subject-matter of the present invention;

FIG. 5 is a longitudinal projection of a third variant of the device which is the subject-matter of the present invention;

FIG. 6 is a longitudinal projection of a fourth variant of the device which is the subject-matter of the present invention, and

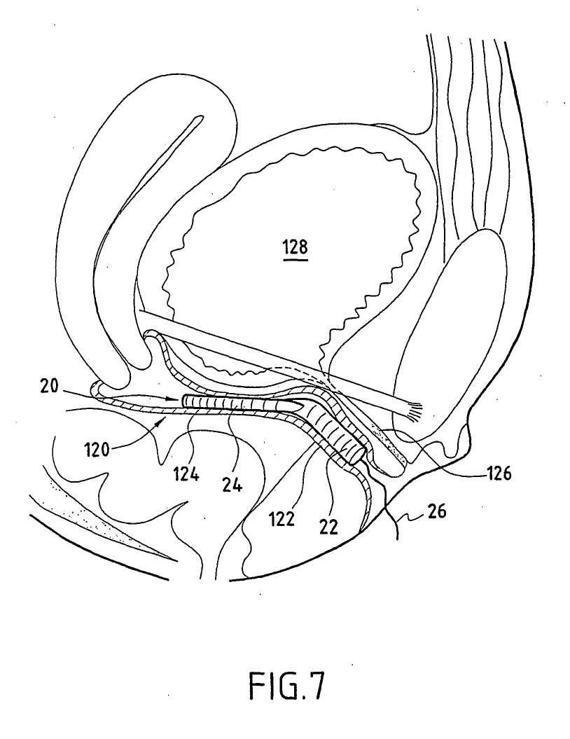

FIG. 7 is a sagittal section of the female vesical and vaginal anatomy with the device according to the invention being positioned.

FIG. 1 shows a device for preventing female urinary stress incontinence which is designated 10 and which comprises three separate elements, that is to say, a treatment element 20, which has a general longitudinal shape and which is to be accommodated in the vagina, and two insertion elements which form insertion means 30 in the form of an applicator used during the positioning of the treatment element 20.

It should be noted that, similarly to sanitary tampons, the applicator 30 can be dispensed with, the treatment element 20 then being inserted manually into the vagina.

The treatment element 20 is in the general form of a periodic intra-vaginal tampon, which is used during menstruation, and comprises a proximal portion 22, a distal portion 24 and preferably a string 26 which acts as means for withdrawing the device.

The adjectives “proximal” and “distal” respectively refer to a portion located in a close and a portion located in a remote manner relative to the hand when the device is positioned in the vagina.

In order to orientate the different dimensions of the device 10 according to three directions perpendicular to each other, the term “length” refers to the largest dimension which is intended to be placed in the direction of the largest dimension of the vagina which extends from the entrance as far as the end of the vagina, the term “height” designates the dimension which is intended to be placed substantially in a vertical manner in the vagina, when the user of the device is upright, and the term “width” designates the dimension which is intended to be placed parallel with the substantially horizontal plane which is formed by the rear or sub-vesical portion of the vagina which forms a hammock-like structure.

The proximal portion 22 extends over a length in the order of 2 cm between a first free end 22a and a second end 22b. The proximal portion 22 is of cylindrical shape generated by revolution having a diameter in the order of 1.5 cm. It will be appreciated that other forms which promote the withdrawal of the device from the vagina are possible for the proximal portion 22.

This proximal portion 22 is produced from materials which ensure both a structure which is substantially non-deformable under pressure and biocompatibility.

To this end, the proximal portion 22 is produced from one or more material(s) which provide(s) it with a substantially hard structure, that is to say, which does not become deformed, in particular which does not flatten, or flattens only slightly, under stress.

The string 26 intended for the removal of the treatment element 20 is attached to the first end 22a of the proximal portion 22 and extends over a length of at least a few centimetres.

The distal portion 24 extends over a length in the order of 3 cm (at least 2 cm and up to 5 cm), extending the second end 22b of the proximal portion 22, is constituted by two identical tubular members 241 and 242 which are of cylindrical shape generated by revolution and which extend in the longitudinal direction.

The junction region between the proximal portion 22 and the distal portion 24 is produced so as to form a progressive transverse change in terms of shape and dimensions.

These tubular members 241 and 242 are produced from materials which ensure both a structure which is deformable under pressure in a reversible manner (with resilient return) and biocompatibility.

For this purpose, the distal portion 24 is produced from one or more materials which impart(s) to the tubular members 241 and 242 a substantially flexible composition, that is to say, a structure which deforms, in particular which flattens, whilst returning to the initial shape thereof after the stress being applied has come to an end.

The tubular members are, for example, produced from a material such as that used for sanitary tampons, that is to say, a material which absorbs liquids, which is woven or nonwoven, with natural fibres (cotton or cellulose) or synthetic fibres, foams, sponges, gels or polymers.

Of course, this distal portion 24 nevertheless ensures a given mechanical strength which is weak and which is far less than the fatigue strength of the proximal portion 22.

In the above, the stress or load being considered relates to the total of all of the pressures applied physiologically to the anterior wall of the vagina. Since they refer to the proximal portion 22 which contributes to the fatigue strength of the front (sub-urethral) portion of the anterior wall of the vagina, these pressures comprise in particular the pressure applied by the abdominal cavity. For the distal portion 24 which contributes to the fatigue strength of the rear (sub-vesical) portion of the anterior wall of the vagina, these pressures comprise in particular the weight of the bladder and the internal pressure of liquid (which vary in accordance with the filling level) as well as the pressure applied by the abdominal cavity.

In the variant illustrated in FIG. 1, the two tubular members 241 and 242 are connected over the length thereof.

The arrangement constituted by these two tubular members imparts a shape, which is wider than it is high, to the distal portion 24. This configuration ensures in particular a wider shape of the distal portion 24 relative to the proximal portion 22, which is important as will be explained below.

The distal portion 24 preferably has a width which is at least 1.5 times the width of the proximal portion 22 and preferably twice this width.

It is further provided that the distal portion 24 has a height which is at most 80% of the height of the proximal portion 22, advantageously between 40 and 60% of the height of the proximal portion 22 and preferably substantially 50% of the height of the proximal portion 22. A distal portion 24 will be used by way of example having a width of between 2.5 and 3.5 cm for a maximum height in the order of 1.5 cm, preferably approximately 1 cm.

The two insertion elements forming the insertion means 30 (or applicator) are constituted by a hollow tube 32 and a pusher 34. The tube 32 forms a housing of circular cross-section for the treatment element 20 before the positioning thereof. The diameter of this housing is substantially equal to or slightly greater than the outside diameter of the proximal portion 22, which is itself in the order of from 1 to 1.5 cm. The two tubular members 241 and 242 have a diameter which is substantially smaller than that of the proximal portion 22, without thereby being smaller than or equal to half of the diameter of the proximal portion 22 owing to the fairly flexible consistency of the distal portion 24.

In this manner, as can be seen in FIG. 2A, the two tubular members 241 and 242 are flattened in the tube 32 before the device is positioned by the user.

It will be appreciated that, in order to position the device, the user presses on the proximal end 34a of the pusher 34 which is carried and guided in a sliding manner inside the housing of the tube 32 in a telescopic manner. In this manner, the treatment element 20 progressively leaves the housing thereof and is positioned in the vagina of the user, as illustrated in FIG. 7.

The first variant illustrated in FIG. 3 provides for the two tubular members 241 and 242 not to be connected over the length thereof, but instead to be separable and move apart from each other at one side and the other of the longitudinal direction, as indicated by the arrows on the left in FIG. 3. This structure is particularly suitable for postmenopausal women who have a distended vagina relative to the vagina of young women, the width of the distal portion 24 being greater with non-connected tubular members and allowing the rear region of the vagina to be supported over a greater lateral extent than when the tubular members 241 and 242 are connected.

FIGS. 4 to 6 illustrate variants of the proximal portion 22, the distal portion 24 being represented in these Figures in an identical manner to that in FIG. 1, but it will be appreciated that it is identical to the distal portion 24 of FIG. 3.

It is provided in the second variant of FIG. 4 for the proximal portion 22′, which is cylindrical and of circular cross-section, to comprise a core 221′ which is surrounded by a casing 222′. The core 221′ is flexible and is produced from the same material as the distal portion 24. The casing 222′ is produced from a hard biocompatible material, that is to say, which is substantially non-deformable under pressure.

The core 221′ is produced from a material which is more flexible than the core 222′, optionally a material which is deformable under pressure in a reversible manner, and preferably from the same material as the distal portion 24.

In the third variant of FIG. 5, the same double structure of the proximal portion 22′ of the second variant is provided in a reversed manner with regard to the features of the materials which constitute the proximal portion 22″: this proximal portion 22″ has a core 221″ which is produced from a hard material surrounded by a flexible casing 222″ which is produced from a biocompatible material.

In the second and third variants, instead of a cylindrical shape having a longitudinal axis as illustrated, the core 221′ or 221″ can have an elongate, for example, oblong, shape.

In the fourth variant illustrated in FIG. 6, the proximal portion 22″′ has a frustoconical shape which widens from the first end 22a towards the second end 22b and the core 222″′, to which the string 26 is connected, is in the shape of an olive.

With reference now to FIG. 7, after the treatment element 20 has been positioned in the vagina 120, the proximal portion 22 extends into the front or sub-urethral portion 122 of the vagina 120 (in the vertical extension of the urethra 126 in the upright position) and the distal portion 24 extends into the rear or sub-vesical portion 124 of the vagina 120 (in the vertical extension of the bladder 128 in the upright position).

Since the distal portion 24 is compressed by the bladder when the bladder is full, the two tubular members are naturally positioned one beside the other substantially in a horizontal plane in order to form a flexible support for the hammock which the rear or sub-vesical portion 124 of the vagina 120 constitutes.

In this manner, it will be appreciated that the device 100 extends over substantially all of the length of the vagina and that it is automatically positioned with the correct angular orientation and in the correct position in terms of depth, whatever the initial orientation of the device when it is positioned by the user.

The present invention relates to a device for countering female urinary stress incontinence which comprises the treatment element 20, the insertion means 30 (hollow tube 32 and pusher 34 or any other equivalent means) being able to be dispensed with without departing from the scope of the present invention.

1. Device (10) for preventing female urinary stress incontinence to be arranged in a longitudinal manner in the vagina, characterised in that it comprises:
a proximal portion (22) which extends between a first free end (22a) and a second end (22b), which is produced so as to be substantially non-deformable under pressure and which is intended to be positioned in the sub-urethral region of the vagina, and
a distal portion (24) which extends the second end (22b) of the proximal portion (22), which is produced from a material which is deformable under pressure in a reversible manner, which is intended to be positioned in the sub-vesical region of the vagina and which has a height which is at most 80% of the height of said proximal portion (22).

2. Device (10) according to claim 1, characterised in that said distal portion (24) has a width which is at least 1.5 times the width of said proximal portion (22).

3. Device (10) according to claim 1 or 2, characterised in that said distal portion (24) has a height of between 40 and 60% of the height of said proximal portion (22) and advantageously in the order of 50% of the height of said proximal portion (22).

4. Device (10) according to any one of claims 1 to 3, characterised in that the distal portion (24) has a width which is greater than the height thereof.

5. Device (10) according to any one of the preceding claims, characterised in that the proximal portion (22) has a length of between 1.5 and 2.5 cm.

6. Device (10) according to any one of the preceding claims, characterised in that the distal portion (24) has a length of between 2 and 5 cm.

7. Device (10) according to any one of the preceding claims, characterised in that the proximal portion (22) has a cylindrical shape of circular cross-section or a frustoconical shape which widens from the first end (22a) towards the second end (22b).

8. Device (10) according to any one of the preceding claims, characterised in that the proximal portion (22″; 22″′) has a core (221″; 221″′) which is produced from a hard material surrounded by a flexible casing (222″; 222″′) which is produced from a biocompatible material.

9. Device (10) according to claim 8, characterised in that said core (221″; 221″′) has, in the longitudinal direction, an elongate shape, either cylindrical with a longitudinal axis and circular cross-section or oblong.

10. Device (10) according to any one of the preceding claims, characterised in that the distal portion (24) is formed by two similar tubular members (241; 242) which are arranged side by side over the length thereof.

11. Device (10) according to any one of the preceding claims, characterised in that the distal portion (24) is produced from foam.

12. Device (10) according to any one of the preceding claims, characterised in that it further comprises withdrawal means (30) for the device which are mounted on the first end of the proximal portion.

13. Device (10) according to claim 10, characterised in that it further comprises an applicator (30) which is formed by a hollow tube (32) which is able to contain the proximal portion (22) and distal portion (24) and a pusher (34) which can slide in a telescopic manner in said tube (32).
